(19) United States
(12) Patent Application Publication
(10) Pub. No.: US 2010/0081891 A1
(43) Pub. Date: Apr. 1, 2010

(54) System And Method For Displaying Detailed Information For A Data Point

(75) Inventors: Hui Wang, San Ramon, CA (US); Li Li, Milpitas, CA (US); Steve Vargas, Sun Valley, CA (US); Robin Boyce, Pleasanton, CA (US); Scott Amundson, Oakland, CA (US); James Ochs, Seattle, WA (US); Tonia Madere, Stockton, CA (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/242,214

(22) Filed: Sep. 30, 2008

(51) Int. Cl. A61B 5/1455; A61B 5/00

(52) U.S. Cl. 600/301; 600/324

(57) ABSTRACT

The present disclosure may describe a system and method for displaying detailed information about a patient at a time selectable from a trend line of historic data. For example, the patient's SpO₂, pulse rate, status, and so forth may be recorded over time. One or more trend lines of the recorded data over time may be displayed, and a user may select a point of interest from the trend line. Detailed data corresponding to that point of interest may then be displayed, for example, next to the trend line or on a separate screen. The detailed information may appear in the form of a pop-up box that does not obscure the trend line. In embodiments, the user may select the data point by moving a cursor or placing a pointing device on the trend line. The detailed information may be displayed automatically or may be activated by further user action.

### BACKGROUND

The present disclosure relates generally to medical devices, and, more particularly, to a pulse oximeter capable of displaying detailed information about a patient's physiological parameters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of healthcare, caregivers (e.g., doctors and other healthcare professionals) often desire to monitor certain physiological characteristics of their patients. Accordingly, a wide variety of monitoring devices have been developed for monitoring many such physiological characteristics. These monitoring devices often provide doctors and other healthcare personnel with information that facilitates provision of the best possible healthcare for their patients. As a result, such monitoring devices have become a perennial feature of modern medicine.

One technique for monitoring physiological characteristics of a patient is commonly referred to as pulse oximetry, and the devices built based upon pulse oximetry techniques are commonly referred to as pulse oximeters. Pulse oximeters may be used to measure and monitor various blood flow characteristics of a patient. For example, a pulse oximeter may be utilized to monitor the blood oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. A photo-plethysmographic waveform, which corresponds to the cyclic attenuation of optical energy through the patient's tissue, may be generated from the detected light. Additionally, one or more of the above physiological characteristics may be calculated based generally upon the amount of light absorbed or scattered. More specifically, the light passed through the tissue may be selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

Generally, the pulse oximeter may display the patient's physiological characteristics as an updating number or as a trend. For example, the patient's current blood oxygen saturation and/or pulse rate may be displayed numerically. In addition, or alternatively, the patient's historical blood oxygen saturation and/or pulse rate over time may be displayed as a trend. In some pulse oximeters, the current and historical data may be displayed on separate screens. If a caregiver wishes to review the patient's historical physical characteristics, the trend(s) may be analyzed visually.

### BRIEF DESCRIPTION OF THE DRAWINGS

Advantages of the disclosure may become apparent upon reading the following detailed description and upon reference to the drawings in which:

FIG. 1 is a perspective view of a pulse oximeter coupled to a multi-parameter patient monitor and a sensor in accordance with embodiments;

FIG. 2 is a block diagram of the pulse oximeter and sensor coupled to a patient in accordance with embodiments;

FIGS. 3-4 are exemplary graphical user interfaces of the pulse oximeter in accordance with embodiments; and

FIG. 5 is a flow chart of an exemplary data display process in accordance with embodiments.

### DETAILED DESCRIPTION

One or more embodiments will be described below. In an effort to provide a concise description of the embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

According to an embodiment a medical monitor, such as a pulse oximeter, may collect and record data regarding a patient's physiological parameters over time. For example, in an embodiment, the pulse oximeter may be coupled to the patient via a sensor which conveys information to the oximeter. The pulse oximeter in turn determines the patient's SpO₂ based on the collected data and saves the determined SpO₂ values over time. In an exemplary embodiment, the pulse oximeter may maintain forty-eight hours of historical SpO₂ values for the patient. The historical data may be displayed on the pulse oximeter or a multi-parameter monitor as a trend line of SpO₂ over time. Other information, such as, for example, the patient's pulse rate or status, may also be determined and recorded along with the SpO₂ values. In accordance with present embodiments, the pulse oximeter may enable a caregiver or user to select a point of interest from the trend line and display detailed information about the patient at or near the time of the selected point of interest. For example, the user may select a point on the trend line, and a box containing a numeric indication of the SpO₂ value, the pulse rate, and the patient's status at the selected time may be displayed on the same or a separate screen.

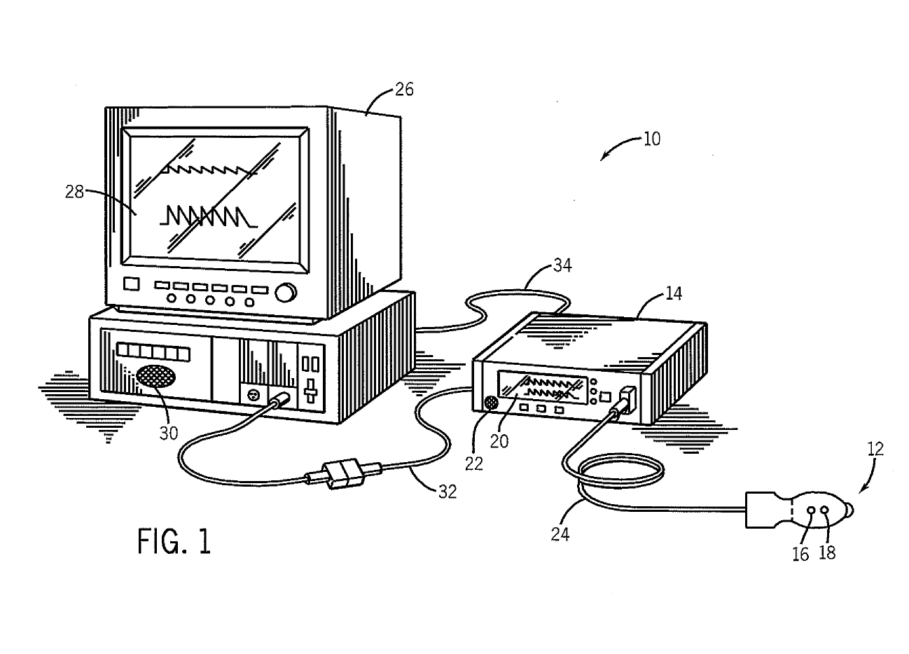

FIG. 1 is a perspective view of such a pulse oximetry system 10 in accordance with an embodiment. The system 10 includes a sensor 12 and a pulse oximetry monitor 14. The sensor 12 includes an emitter 16 for emitting light at certain wavelengths into a patient's tissue and a detector 18 for detecting the light after it is reflected and/or absorbed by the patient's tissue. The monitor 14 may be capable of calculating physiological characteristics received from the sensor 12 relating to light emission and detection. Further, the monitor 14 includes a display 20 capable of displaying the physiological characteristics, historical trends of the physiological characteristics, other information about the system, and/or alarm indications. The monitor 14 also includes a speaker 22 to provide an audible alarm in the event that the patient's physiological characteristics exceed a threshold. The sensor 12 is communicatively coupled to the monitor 14 via a cable 24. However, in other embodiments a wireless transmission device or the like may be utilized instead of or in addition to the cable 24.

In the illustrated embodiment, the pulse oximetry system 10 also includes a multi-parameter patient monitor 26. In addition to the monitor 14, or alternatively, the multi-parameter patient monitor 26 may be capable of calculating physiological characteristics and providing a central display 28 for information from the monitor 14 and from other medical monitoring devices or systems. For example, the multi-parameter patient monitor 26 may display a patient's SpO₂ and pulse rate information from the monitor 14 and blood pressure from a blood pressure monitor on the display 28. Additionally, the multi-parameter patient monitor 26 may indicate an alarm condition via the display 28 and/or a speaker 30 if the patient's physiological characteristics are found to be outside of the normal range. The monitor 14 may be communicatively coupled to the multi-parameter patient monitor 26 via a cable 32 or 34 coupled to a sensor input port or a digital communications port, respectively. In addition, the monitor 14 and/or the multi-parameter patient monitor 26 may be connected to a network to enable the sharing of information with servers or other workstations.

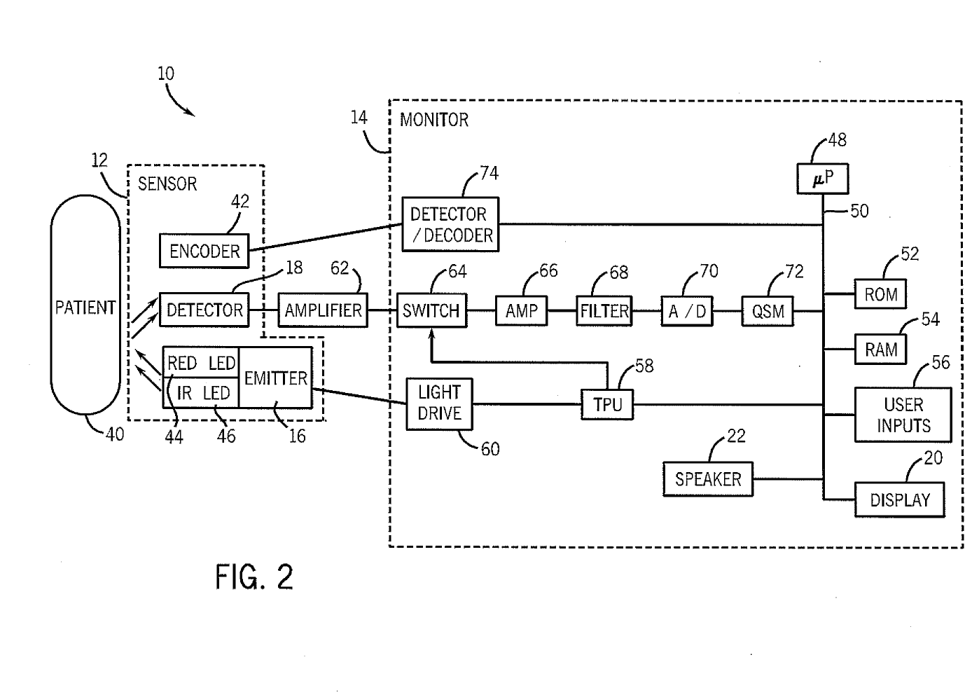

FIG. 2 is a block diagram of the exemplary pulse oximetly system 10 of FIG. 1 coupled to a patient 40 in accordance with an embodiment. One such pulse oximeter that may be used in the implementation of the present disclosure is the OxiMax® N-600x™ available from Nellcor Puritan Bennett LLC, but the following discussion may be applied to other pulse oximeters and medical devices. Specifically, certain components of the sensor 12 and the monitor 14 are illustrated in FIG. 2. The sensor 12 may include the emitter 16, the detector 18, and an encoder 42. It should be noted that the emitter 16 may be capable of emitting at least two wavelengths of light e.g., RED and IR, into a patient's tissue 40. Hence, the emitter 16 may include a RED LED 44 and an IR LED 46 for emitting light into the patient's tissue 40 at the wavelengths used to calculate the patient's physiological characteristics.

In embodiments, the RED wavelength may be between about 600 nm and about 700 nm, and the IR wavelength may be between about 800 nm and about 1000 nm. Alternative light sources may be used in other embodiments. For example, a single wide-spectrum light source may be used, and the detector 18 may be capable of detecting certain wavelengths of light. In another example, the detector 18 may detect a wide spectrum of wavelengths of light, and the monitor 14 may process only those wavelengths which are of interest. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In an embodiment the detector 18 may be capable of detecting the intensity of light at the RED and IR wavelengths. In operation, light enters the detector 18 after passing through the patient's tissue 40. The detector 18 may convert the intensity of the received light into an electrical signal. The light intensity may be directly related to the absorbance and/or reflectance of light in the tissue 40. That is, when more light at a certain wavelength is absorbed or reflected, less light of that wavelength is typically received from the tissue by the detector 18. After converting the received light to an electrical signal, the detector 18 may send the signal to the monitor 14, where physiological characteristics may be calculated based at least in part on the absorption of the RED and IR wavelengths in the patient's tissue 40.

According to an embodiment, the encoder 42 may contain information about the sensor 12, such as what type of sensor it is (e.g., whether the sensor is intended for placement on a forehead or digit) and the wavelengths of light emitted by the emitter 16. This information may allow the monitor 14 to select appropriate algorithms and/or calibration coefficients for calculating the patient's physiological characteristics. The encoder 42 may, for instance, be a coded resistor which stores values corresponding to the type of the sensor 12 and/or the wavelengths of light emitted by the emitter 16. These coded values may be communicated to the monitor 14, which determines how to calculate the patient's physiological characteristics. In another embodiment, the encoder 42 may be a memory on which one or more of the following information may be stored for communication to the monitor 14: the type of the sensor 12; the wavelengths of light emitted by the emitter 16; and the proper calibration coefficients and/or algorithms to be used for calculating the patient's physiological characteristics. Exemplary pulse oximetry sensors capable of cooperating with pulse oximetry monitors are the OxiMax® sensors available from Nellcor Puritan Bennett LLC.

According to an embodiment, signals from the detector 18 and the encoder 42 may be transmitted to the monitor 14. The monitor 14 generally may include processors 48 connected to an internal bus 50. Also connected to the bus may be a read-only memory (ROM) 52, a random access memory (RAM) 54, user inputs 56, the display 20, or the speaker 22. A time processing unit (TPU) 58 may provide timing control signals to a light drive circuitry 60 which controls when the emitter 16 is illuminated and the multiplexed timing for the RED LED 44 and the IR LED 46. The TPU 58 control the gating-in of signals from detector 18 through an amplifier 62 and a switching circuit 64. These signals may be sampled at the proper time, depending upon which light source is illuminated. The received signal from the detector 18 may be passed through an amplifier 66, a low pass filter 68, and an analog-to-digital converter 70. The digital data may then be stored in a queued serial module (QSM) 72 for later downloading to the RAM 54 as the QSM 72 fills up. In one embodiment, there may be multiple separate parallel paths having the amplifier 66, the filter 68, and the A/D converter 70 for multiple light wavelengths or spectra received.

According to an embodiment, the processor(s) 48 may determine the patient's physiological characteristics, such as SpO₂ and pulse rate, using various algorithms and/or look-up tables based generally on the value of the received signals corresponding to the light received by the detector 18. Signals corresponding to information about the sensor 12 may be transmitted from the encoder 42 to a decoder 74. The decoder 74 may translate these signals to enable the microprocessor to determine the proper method for calculating the patient's physiological characteristics, for example, based generally on algorithms or look-up tables stored in the ROM 52. In addition, or alternatively, the encoder 42 may contain the algorithms or look-up tables for calculating the patient's physiological characteristics. The user inputs 56 may be used to select historical data points for measured physiological characteristics on the monitor 14, as described below. In certain embodiments, the display 20 may exhibit additional detailed information about one or more of the patient's physiological parameters when a historic data point is selected.

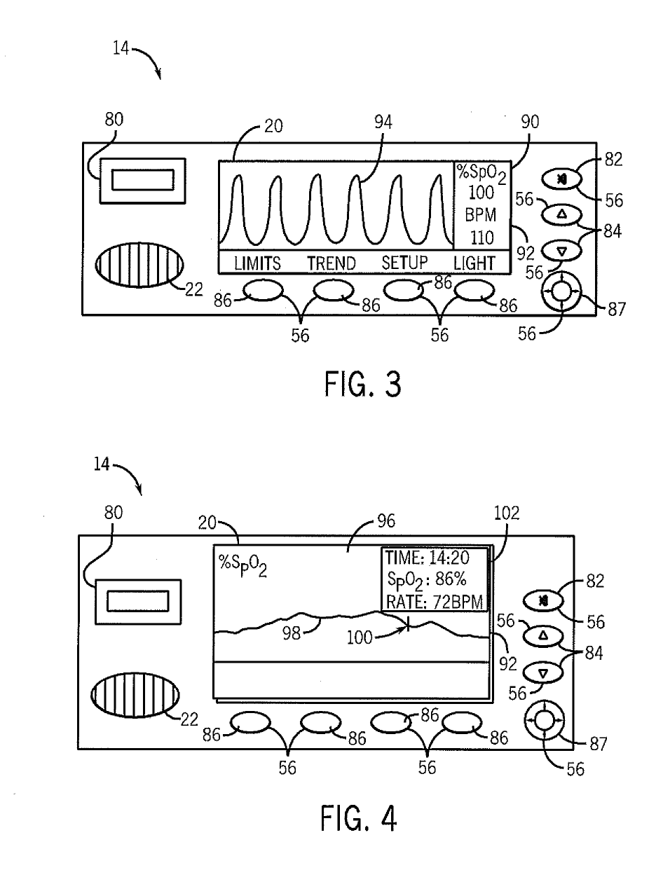

FIG. 3 illustrates an embodiment of a monitor 14 for use in the system 10 (FIG. 1). The monitor 14 may generally include the display 20, the speaker 22, the user inputs 56, and a communication port 80 for coupling the sensor 12 (FIG. 2) to the monitor 14. The user inputs 56 may enable the caregiver to control the monitor 14 and change settings. For example, an alarm silence button 82 may enable the caregiver to silence an audible alarm (e.g., when the patient is being cared for), and volume buttons 84 may enable the caregiver to adjust the volume of the alarm and/or any other indicators emitted from the speaker 22.

In addition, soft keys 86 may correspond to variable functions, as displayed on the display 20. The soft keys 86 may provide access to further data and/or setting displays. For example, the soft keys 86 provided on the display 20 may enable the caregiver to scroll through data points, activate and/or deactivate an additional data display, see and/or change alarm thresholds, view different trend data, change characteristics of the display 20, turn a backlight on or off, or perform other functions. The monitor 14 may further include a pointing device 87 to enable the user to move a virtual indicator on the display 20. The pointing device 87 may include, for example, a joystick, a trackball, an eraser mouse, a point-and-click mouse, or another multi-directional interface device. In some embodiments, the display 20 may be a touch-sensitive screen which operates as an additional user input 56.

According to an embodiment, the display 20 maybe capable of displaying multiple screens selectable, for example, via the soft keys 86. In an exemplary embodiment, illustrated in FIG. 3, a default operating screen 88 may be displayed during standard operation of the monitor 14 (i.e., during patient monitoring). For example, the default operating screen 88 may show an SpO₂ value 90, a pulse rate 92, and/or a plethysmographic waveform 94. In an embodiment, illustrated in FIG. 4, another screen 96 capable of being shown on the display 20 may display one or more trend lines 98 illustrating historical data, such as, for example, the patient's measured SpO₂, pulse rate, or other physiological parameters. The trend line screen 96 may be accessible from the default operating screen 88, for example, via the soft keys 86.

As described, the embodiment of a screen 96 may illustrate the historic data trend line 98. This trend line 98 may be selectable overall via the user inputs 56, such as the soft keys 86, the pointing device 87, the display 20, and so forth. In addition, data points 100 making up the trend line 98 may be selectable via the user inputs 56. For example, a user may be able to select a desired point 100 on the trend line 98 corresponding to an event of interest, such as the beginning or end of a treatment, or a specific time. The user may select the data point 100, for example, by positioning an indicator (e.g., an arrow) for the pointing device 87 over the trend line 98 or moving a scrolling indicator (e.g., a cursor) along the tend line 98 with the soft keys 86. Additional information about the patient 40 corresponding to the selected data point 100 may then be illustrated, such as in a pop-up box 102 displayed on the trend line screen 96.

According to an embodiment, the additional information may include, for example, the patient's SpO₂ value, pulse rate, identifying information, saturation pattern detection index (e.g., an indication of repetitive reductions in airflow through the upper airway and into the lungs), and/or status indicator; the monitor's percent modulation and/or alarm limits; the sensor's artifacts and/or status; a time stamp for the selected data point 100; or any other pertinent information collected concurrently with or in close temporal proximity to the selected data point 100. In addition, the patient's status at the time of the selected data point 100 may be determined generally based on the information collected about the patient at or near that time. That is, if the patient's physiological parameters collected at the time of the selected data point 100 were within alarm limits, the patient's status may indicate “HEALTHY” or a graphical symbol may be displayed to indicate a healthy state. Similarly, if the physiological parameters were outside alarm limits, the status indicator may indicate “<NOT HEALTHY>”, or other indication. In an embodiment the pop-up box 102 and/or the displayed detailed information may be color-coded to indicate the patient's status or to provide additional information regarding the selected data point 100. For example, if the patient's status was “HEALTHY,” the pop-up box 102 and/or the displayed information may be illustrated in green, whereas the pop-up box 102 may be illustrated in red if the patient's status was “<NOT HEALTHY>.”

In an embodiment, the pop-up box 102 may be activated and/or deactivated automatically when the data point 100 is selected. In another embodiment, an additional action may activate/deactivate the pop-up box 102. For example, the user may position the virtual indicator for the pointing device 87 over the trend line 98 and press a button to activate the pop-up box 102. In another embodiment, the user may position the scrolling indicator at the desired data point 100 and press one of the soft keys 86 to activate the pop-up box 102. Furthermore, the additional information may be displayed on another screen capable of being shown on the display 20 rather than, or in addition to, being displayed in the pop-up box 102.

Users of the monitor 14 may be able to customize the detailed information that is shown based on the information that is most relevant to them. For example, a nurse may be interested in reviewing only the patient's SpO₂ and pulse rate, whereas a doctor may configure the pop-tip box 102 to display much more detailed information. In another embodiment, the pop-up box 102 may display a truncated list of detailed information which is expandable based on the user's actions. For example, the user may position the virtual indicator for the pointing device 87 over the trend line 98 to see some of the patient's detailed information at the selected data point 100, and then additional information may be displayed if the user presses a button or leaves the virtual indicator positioned over the same data point 100 for an extended period of time (e.g., three seconds).

According to an embodiment, the user may be able to select items displayed in the pop-up box 102 to access additional information about the selected item. In an embodiment, the user may be viewing the patient's historical SpO₂ data trend line 98. The user may activate the pop-up box 102 at a desired time point and select the pulse rate from the pop-up box 102. The patient's historical pulse rate data trend line 98 may then be displayed rather than, or in addition to, the SpO₂ data trend line 98. In another embodiment, the user may be able to adjust settings for the monitor 14 by selecting setting information in the pop-up box 102. For example, in an embodiment, the user may select a data point from the SpO₂ trend line 98 and select the alarm limits from the pop-up box 102. Another screen may then displayed at which the user may change the alarm limits, or the alarm limits may be displayed on the trend line screen 96.

In some circumstances, it may be desirable to see detailed information about a certain data point 100 without obstructing the remainder of the trend line 98. Accordingly, in some embodiments, the pop-up box 102 may automatically appear at a position on the screen 96 where the box 102 does not obscure any part of the trend line 98. In these embodiments, the position of the pop-up box 102 may change as the user selects different data points 100 along the trend line 98. In another embodiment, the pop-tip box 102 may appear at one designated location on the trend line screen 96. The designated location may be dedicated to the pop-up box 102 such that the trend line 98 is not displayed in that area of the screen 96 (e.g., a top corner of the screen 96 may be reserved for display of the detailed information).

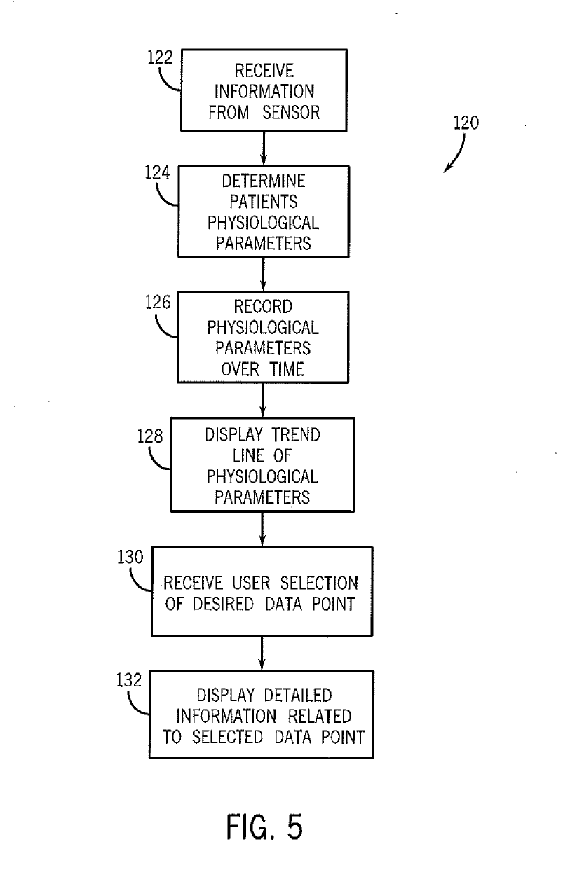

FIG. 5 illustrates an embodiment of a process 120 by which the monitor 14 may display detailed information about a patient from a selected point in time. In the illustrated embodiment, the monitor 14 may receive information from the sensor 12 coupled to the patient 40 (block 122). Based on the received information, the monitor 14 may determine the patient's physiological parameters and display information on the display 20, as shown in the embodiment illustrated in FIG. 3 (block 124). In addition, the monitor 14 may record the patient's physiological parameters, status, and any other relevant information over time (block 126). In some embodiments, the historical physiological data may be illustrated as a trend line along with the current information. In other embodiments, the caregiver may choose to view the trend line screen 96, for example, by selecting one of the soft keys 86 from the default screen 88. The monitor 14 may then display the one or more trend lines 98 on the display 20 (block 128).

According to an embodiment, the monitor 14 may subsequently receive user input indicating selection of a desired data point 100 on the trend line 98 (block 130). In an embodiment, the caregiver may select one of the soft keys 86 to enable a cursor capable of moving along the trend line 98, and then the caregiver may move the cursor along the trend line 98 to a data point of interest, for example, using the soft keys 86. In another embodiment, the caregiver may select a data point of interest by placing the virtual indicator for the pointing device 87 over the desired point and pressing a button. In a further embodiment, the caregiver may select the historical data point 100 by touching the display 20 at the desired location on the trend line 98. Upon receiving user selection of the point of interest, the monitor may display the pop-up box 102, as shown in the embodiment illustrated in FIG. 4, or an additional screen containing additional detailed information about the patient at the time of the selected data point (block 130). When the caregiver has finished reading the detailed information, the pop-up box 102 may be deactivated by pressing the same or another soft key 86. The caregiver may then return to the default operating screen 88 by choosing the appropriate soft key 86 to escape from the trend line screen 96.

While only certain features have been illustrated and described herein, many modifications and changes will occur to those skilled in the art. It is, therefore, to be understood that the appended claims are intended to cover all such modifications and changes as fall within their true spirit.

What is claimed is:

1. A method, comprising:
displaying a trend line of physiological data on a first screen on a display of a physiological monitor;
receiving a user selection of a point on the trend line;
displaying information about the user-selected point on the display.

2. The method of claim 1, comprising determining the physiological data based on signals received from a physiological sensor.

3. The method of claim 1, wherein displaying information about the user-selected point comprises displaying a pop-up box on the first screen.

4. The method of claim 3, comprising displaying the pop-up box on the first screen such that no part of the trend line is obscured.

5. The method of claim 1, wherein displaying information about the user-selected point comprises displaying a second screen.

6. The method of claim 1, wherein the physiological data comprises a blood oxygen saturation and/or a pulse rate.

7. The method of claim 1, wherein the information about the user-selected point comprises a time stamp, a patient identification, a saturation pattern detection index, a patient status indicator, a monitor percent modulation, one or more monitor alarm limits, a sensor artifact indication and/or a sensor status indication.

8. A method, comprising:
selecting a point on a displayed trend line of physiological data to activate a display of additional information related to the trend line.

9. The method of claim 8, wherein selecting the point comprises moving an indicator along the trend line via one or more directional keys.

10. The method of claim 8, wherein selecting the point comprises positioning a virtual indicator representing a pointing device over the trend line.

11. The method of claim 8, wherein selecting the point comprises touching a display on which the trend line is displayed.

12. The method of claim 8, wherein selecting the point comprises placing an indicator at the point and sending a signal to indicate that the point has been selected.

13. The method of claim 8, wherein the physiological data comprises a blood oxygen saturation and/or a pulse rate.

14. The method of claim 85 wherein the information about the user-selected point comprises a time stamp, a patient identification, a saturation pattern detection index, a patient status indicator, a monitor percent modulation, one or more monitor alarm limits, a sensor artifact indication and/or a sensor status indication.

15. One or more tangible, machine-readable media comprising code which, if executed by a processor, cause the processor to render on a display of a physiological monitor:
a trend line of physiological data; and
additional information about a point on the trend lie upon user selection of the point.

16. The one or more tangible, machine-readable media of claim 15, comprising code which, if executed by a processor, cause the processor to render the additional information about the point adjacent to and not overlapping the rendered trend line.

17. The one or more tangible, machine-readable media of claim 15, comprising code which, if executed by a processor, cause the processor to render the additional information about the point in place of the trend line.

18. A physiological monitor, comprising:
a user input interface;
a display; and
a processor capable of at least displaying a trend line of physiological data on the display and displaying additional information about a point on the trend line upon selection of the point via the user input interface.

19. The monitor of claim 18, wherein the user input interface comprises a joystick, a trackball, an eraser mouse, and/or a point-and-click-mouse.

20. The monitor of claim 18, wherein the user input interface comprises the display.
